(19) United States
(12) Patent Application Publication
(10) Pub. No.: US 2024/0238595 A1
(43) Pub. Date: Jul. 18, 2024

(54) TIME AVERAGED SIGNAL REDUCTION SCHEME USING FREQUENCY DITHERING FOR REAL-TIME STIMULATION ARTIFACT REMOVAL

(71) Applicant: Korea Advanced Institute of Science and Technology, Daejeon (KR)

(72) Inventors: Chul Kim, Daejeon (KR); Geunchang Seong, Daejeon (KR)

(73) Assignee: Korea Advanced Institute of Science and Technology, Daejeon (KR)

(21) Appl. No.: 18/532,432

(22) Filed: Dec. 7, 2023

(30) Foreign Application Priority Data
Dec. 7, 2022 (KR) 10-2022-0169623
Nov. 20, 2023 (KR) 10-2023-0160603

(51) Int. Cl. A61N 1/36

(52) U.S. Cl. CPC A61N 1/36171

(57) ABSTRACT

The present disclosure relates to a technology for attenuating an average value of signals in a frequency dithering time domain, which may be used in a method of removing stimulus artifacts in real time. A dithering range of a stimulus frequency for generating a stimulus can be set, and the stimulus frequency can be controlled to be changed within the dithering range.

### CROSS-REFERENCES TO RELATED APPLICATIONS

This application is based on and claims priority under 35 U.S.C. 119 to Korean Patent Application Nos. 10-2022-0169623, filed on Dec. 7, 2022 and 10-2023-0160603, filed on Nov. 20, 2023 in the Korean intellectual property office, the disclosure of which is herein incorporated by reference in their entirety.

### TECHNICAL FIELD

The following description relates to a technology for removing artifacts that occur due to an electric stimulus.

### BACKGROUND OF THE DISCLOSURE

For an implementation, such as a closed loop brain-machine connection or an electroceutical for an in-vitro or in-vivo electric stimulus, it is necessary to measure a bio (or neural) signal in an electric stimulus environment. A method of stimulating a nerve tissue by using an electric stimulus is used a lot as a method of investigating connectivity within a nerve tissue and the treatment of degenerative brain diseases or a treatment method for reducing the symptoms of degenerative brain diseases.

It is very important to accurately measure an electrical reaction of a nerve cell in a portion to which an electric stimulus has been applied simultaneously with the stimulus in analyzing effects of the stimulus. However, there is a great difficulty in analyzing a recording signal because the stimulus signal acts as artifacts for the recording signal. That is, artifacts that generate the electric stimulus hinder the reading of a bio signal.

In order to solve such a problem, there is used a method of measuring a bio signal after waiting until artifacts disappear after the electric stimulus. Furthermore, as an example of a technology for removing artifacts, a technology for removing stimulus artifacts by using a sine wave stimulus has been disclosed in Korean Patent No. 10-1659149 (issued on Sep. 13, 2016).

Recently, a technology for removing stimulus artifacts in real time using a time domain average method of removing artifacts from hardware in real time is used. In the technology for removing stimulus artifacts in real time using the time domain average method, in order to obtain a repeated stimulus artifact signal, signals in an interval including stimulus artifacts are averaged in the time domain.

However, there is a problem in that even a bio signal having the same (or multiple) frequency as a stimulus frequency is removed when artifacts are removed because the bio signal is also included in an average value (or calculated artifacts).

### SUMMARY

In a technology for averaging signals within a stimulus artifact interval in the time domain, a stimulator generates a stimulus signal having always the same frequency in order to obtain a stimulus artifact signal. Accordingly, there is a problem in that a bio signal having the same frequency as a stimulus frequency is removed along with artifacts because the bio signal is also included in a time domain average value.

Embodiments of the present disclosure provide a method capable of maintaining effects of a stimulus by changing a stimulus frequency within an intended dithering range and overcoming a problem in that a bio signal having the same frequency as the stimulus frequency is also removed when artifacts are removed.

There is provided a method of removing stimulus artifacts by a computer device including at least one processor, including setting, by the at least one processor, a dithering range of a stimulus frequency for generating a stimulus and controlling, by the at least one processor, the stimulus frequency to be changed within the dithering range.

According to an aspect, the controlling of the stimulus frequency may include changing the stimulus frequency by changing variables related to stimulation duration.

According to another aspect, the controlling of the stimulus frequency may include controlling the stimulus frequency to be changed in a predetermined range on the basis of a center frequency by changing an adjustment signal for generating a signal that turns on/off switches of a stimulus generation circuit as a stimulus signal.

According to still another aspect, the method may further include setting, by the at least one processor, a center frequency that is a dithering criterion.

According to still another aspect, the setting of the dithering range may include setting the dithering range of the stimulus frequency as a predetermined range on the basis of the center frequency.

According to still another aspect, when the stimulus frequency is dithered, the center frequency may become the dithering criterion so that a frequency dithered within a predetermined range is placed in the stimulus frequency.

According to still another aspect, the setting of the center frequency may include setting at least one of stimulation parameters including a form and length of the stimulus.

According to still another aspect, the length of the stimulus may be set for each phase and may include an inter-phase gap.

There is provided a computer device including at least one processor configured to execute a computer-readable instruction. The at least one processor includes a dithering range setting unit configured to set a dithering range of a stimulus frequency for generating a stimulus and a stimulus characteristic setting unit configured to set variables related to the stimulus so that the stimulus frequency is changed within the dithering range.

According to embodiments of the present disclosure, it is possible to maintain effects of a stimulus by controlling a stimulus frequency to be changed within an intended dithering range and also to effectively solve a problem in that a bio signal having the same frequency as the stimulus frequency is removed when artifacts are removed.

### DESCRIPTION OF THE DRAWINGS

FIG. 1 is a block diagram for describing an example of internal components of a computer device in an embodiment of the present disclosure.

FIG. 2 is a block diagram illustrating an example of components which may be included in the processor of the computer device according to an embodiment of the present disclosure.

FIG. 3 illustrates an example of an H-bridge circuit that is constructed as a stimulator controller.

FIG. 4 illustrates an example of stimulus variables that are related to time.

FIG. 5 is a flowchart illustrating an example of a method of removing stimulus artifacts, which may be performed by the computer device according to an embodiment of the present disclosure.

FIG. 6 illustrates an example of the results of Verilog simulation experiments.

FIGS. 7 to 9 illustrate examples of the results of MATLAB simulations.

### DETAILED DESCRIPTION

Hereinafter, embodiments of the present disclosure are described in detail with reference to the accompanying drawings.

Embodiments of the present disclosure relate to a technology for removing artifacts occurring due to an electric stimulus.

According to embodiments including contents that are specifically disclosed in this specification, a specific frequency attenuation phenomenon occurring in a method of removing stimulus artifacts using a time domain average value can be effectively avoided through dithering for a stimulus frequency.

An apparatus for removing stimulus artifacts according to embodiments of the present disclosure may be implemented by at least one computer device. A method of removing stimulus artifacts according to embodiments of the present disclosure may be performed through at least one computer device included in the apparatus for removing stimulus artifacts. In this case, a computer program according to an embodiment of the present disclosure may be installed and driven in the computer device. The computer device may perform the method of removing stimulus artifacts according to embodiments of the present disclosure under the control of the computer program. The computer program may be stored in a computer-readable recording medium in order to execute the method of removing stimulus artifacts in a computer in combination with the computer device.

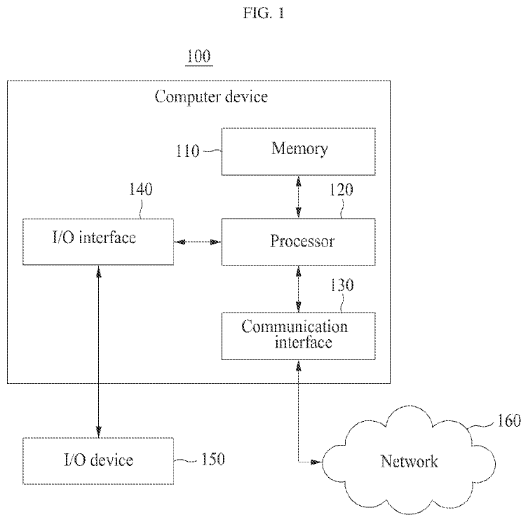

FIG. 1 is a block diagram for describing an example of internal components of a computer device in an embodiment of the present disclosure. For example, the apparatus for removing stimulus artifacts according to embodiments of the present disclosure may be implemented by a computer device 100 illustrated in FIG. 1.

As illustrated in FIG. 1, the computer device 100 according to embodiments of the present disclosure is a component for executing the method of removing stimulus artifacts, and may include memory 110, a processor 120, a communication interface 130, and an input/output (I/O) interface 140.

The memory 110 is a computer-readable recording medium, and may include permanent mass storage devices, such as random access memory (RAM), read only memory (ROM), and a disk drive. In this case, the permanent mass storage device, such as ROM and a disk drive, may be included in the computer device 100 as a permanent storage device separated from the memory 110. Furthermore, an operating system and at least one program code may be stored in the memory 110. Such software components may be loaded onto the memory 110 from a computer-readable recording medium separated from the memory 110. Such a separate computer-readable recording medium may include computer-readable recording media, such as a floppy drive, a disk, a tape, a DVD/CD-ROM drive, and a memory card. In another embodiment, the software components may be loaded onto the memory 110 through the communication interface 130 not a computer-readable recording medium. For example, the software components may be loaded onto the memory 110 of the computer device 100 based on a computer program installed by files that are received over a network 160.

The processor 120 may be configured to process instructions of a computer program by performing basic arithmetic, logic, and input/output (I/O) operations. The instructions may be provided to the processor 120 by the memory 110 or the communication interface 130. For example, the processor 120 may be configured to execute received instructions based on a program code that has been stored in a recording device, such as the memory 110.

The communication interface 130 may provide a function for enabling the computer device 100 to communicate with other devices over the network 160. For example, a request, an instruction, data, or a file that is generated by the processor 120 of the computer device 100 based on a program code that has been stored in a recording device, such as the memory 110, may be transferred to other devices over the network 160 under the control of the communication interface 130. Inversely, a signal, an instruction, data, or a file from another device may be received by the computer device 100 through the communication interface 130 of the computer device 100 over the network 160. A signal, an instruction, a file that is received through the communication interface 130 may be transmitted to the processor 120 or the memory 110. A file that is received through the communication interface 130 may be stored in a storage medium (e.g., the aforementioned permanent storage device) which may be further included in the computer device 100.

The communication method is not limited, and may include short-distance wired/wireless communication between devices, in addition to communication methods using communication networks (e.g., a mobile communication network, wired Internet, wireless Internet, and a broadcasting network) which may be included in the network 160. For example, the network 160 may include one or more arbitrary networks of a personal area network (PAN), a local area network (LAN), a campus area network (CAN), a metropolitan area network (MAN), a wide area network (WAN), a broadband network (BBN), and the Internet. Furthermore, the network 160 may include one or more of network topologies, including a bus network, a star network, a ring network, a mesh network, a star-bus network, and a tree or hierarchical network, but is not limited thereto.

The I/O interface 140 may be means for an interface with an VO device 150. For example, the input device may include a device, such as a microphone, a keyboard, a camera, or a mouse. The output device may include a device, such as a display or a speaker. Furthermore, for example, the I/O interface 140 may be means for an interface with a device in which functions for an input and an output have been integrated into one, such as a touch screen. The I/O device 150, together with the computer device 100, may be configured as a single device.

Furthermore, in other embodiments, the computer device 100 may include components greater or smaller than the components of FIG. 1. However, it is not necessary to clearly illustrate most of conventional components. For example, the computer device 100 may be implemented to include at least some of the I/O devices 150 or may further include other components, such as a transceiver, a camera, various sensors, and a database.

Hereinafter, detailed embodiments of a technology for attenuating an average value of signals in a frequency dithering time domain, which may be used in a method of removing stimulus artifacts in real time, are described.

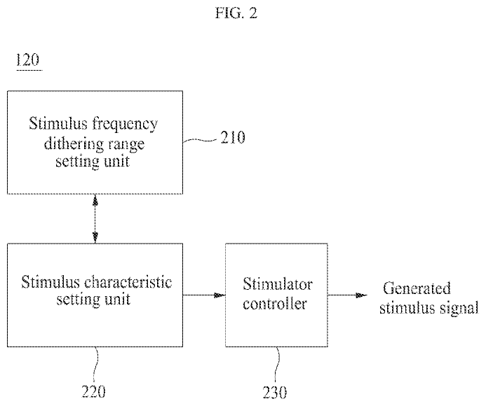

FIG. 2 is a block diagram illustrating an example of components which may be included in the processor of the computer device according to an embodiment of the present disclosure.

The computer device 100 according to the present embodiment may provide a targeted service (e.g., a bio signal measurement service) with a client through a dedicated application that is installed in the client or a connection to a web/mobile site related to the computer device 100.

The processor 120 of the computer device 100 is a component for performing the method of removing stimulus artifacts, which will be described later, and may include a stimulus frequency dithering range setting unit 210, a stimulus characteristic setting unit. 220, and a stimulator controller 230 as illustrated in FIG. 2. According to an embodiment, the components of the processor 120 may be selectively included or excluded in the processor 120. Furthermore, according to an embodiment, the components of the processor 120 may be separated or merged for the expression of a function of the processor 120.

The processor 120 and the components of the processor 120 may control the computer device 100 so that the computer device 100 performs steps included in the method of removing stimulus artifacts. For example, the processor 120 and the components of the processor 120 may be implemented to execute an instruction according to a code of an operating system and a code of at least one program, which is included in the memory 110.

In this case, the components of the processor 120 may be the expressions of different functions that are performed by the processor 120 in response to an instruction that is provided by a program code stored in the computer device 100. For example, the stimulus frequency dithering range setting unit 210 may be used as a functional expression of the processor 120 that controls the computer device 100 in response to the instruction so that the computer device 100 performs a stimulus frequency dithering range.

The processor 120 may read a required instruction from the memory 110 onto which instructions related to control of the computer device 100 have been loaded. The read instruction may include an instruction for controlling the processor 120 to execute the method of removing stimulus artifacts.

The stimulus characteristic setting unit 220 plays a role of setting which stimulus will be generated. The setting may include the setting of a stimulus frequency and the setting of a stimulus waveform. For example, the stimulus characteristic setting unit 220 may set the center frequency of a stimulus frequency, that is, a criterion, when stimulus frequency dithering operates for the setting of the stimulus frequency. In this case, the center frequency becomes a criterion when the stimulus frequency is dithered, so that a frequency dithered in a predetermined range is placed at the center frequency of the stimulus frequency. Furthermore, the stimulus characteristic setting unit 220 may set parameters related to a stimulus, such as a form of the stimulus or the length of the stimulus, for the setting of the stimulus waveform. The form of the stimulus may be a mono-phasic waveform, a bi-phasic waveform, a bi-phasic square wave, or a triangle wave. The length of the stimulus may be differently set for each phase, and may also include an inter-phase gap.

The stimulus frequency dithering range setting unit 210 may determine within which range stimulus frequency dithering will be performed on the basis of the center frequency of a stimulus frequency, which has been set by the stimulus characteristic setting unit 220, through the setting of a stimulus frequency, and may transmit a corresponding signal to the stimulus characteristic setting unit 220. The stimulus frequency dithering range setting unit 210 may generate a stimulus generation signal in which the stimulus frequency dithering is changed about within a set range (e.g., 10%) in the center frequency, and may change a variation range of the stimulus frequency dithering depending on the setting and construction of a stimulus frequency.

The stimulator controller 230 actually generates a stimulus based on a stimulus generation signal and a stimulus waveform setting value. The generated stimulus may be a voltage stimulus, a current stimulus, or a charge stimulus, and a different method may be used depending on its construction.

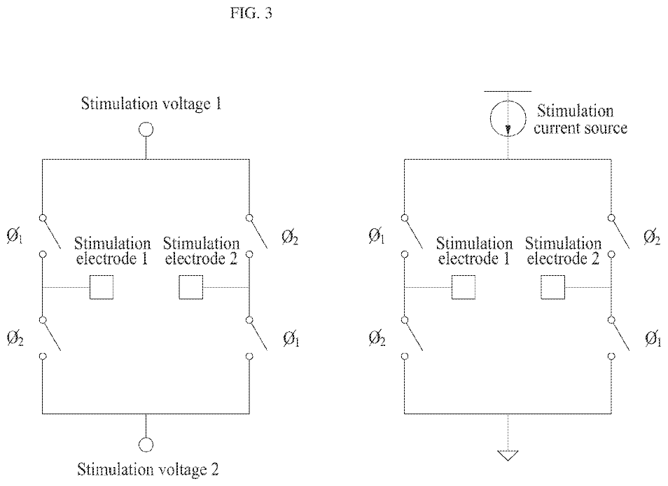

FIG. 3 illustrates an example of an H-bridge circuit.

In order to generate a mono-phasic or bi-phasic square wave, as illustrated in FIG. 3, the H-bridge circuit is constructed as the stimulator controller 230. The stimulus characteristic setting unit 220 may generate a signal that turns on/off the switches of the H-bridge circuit. The stimulus frequency dithering range setting unit 210 may change an adjustment signal of the stimulator controller 230, which is generated by the stimulus characteristic setting unit 220, so that a stimulus frequency is changed in a predetermined range on the basis of the center frequency of the stimulus frequency.

A method of generating, by the stimulus characteristic setting unit 220, a stimulator control signal that adjusts a desired waveform (e.g., a mono-phasic or bi phasic wave), a stimulus frequency, the length of an inter-phase gap, or the length of a stimulus is as follows.

For example, a high frequency clock signal may be generated through an oscillator (e.g., a crystal oscillator or a ring oscillator). On/off timing of each of the switches of the H-bridge circuit may be determined by using the cycle of the high frequency clock signal as a basis unit. For example, assuming that a clock signal of 100 MHz has been generated, each value of a counter may have a 10 ns time by connecting the clock signal to the counter.

In the H-bridge circuit of FIG. 3, assuming that the switch is turned on whenever (the level of a stimulus) Φ is high, a bi-phasic square wave of 10 MHz or more can be generated by changing (the level of a stimulus) Φ₁ from “high” to “low” or “low” to “high” whenever the number of the counter becomes 5 and initializing the counter whenever (the level of the stimulus) Φ is changed while changing (the level of a stimulus) Φ₂ contrary to (the level of the stimulus) Φ₁.

If the method of generating a stimulator control signal is used, a stimulus can be adjusted based on several variables.

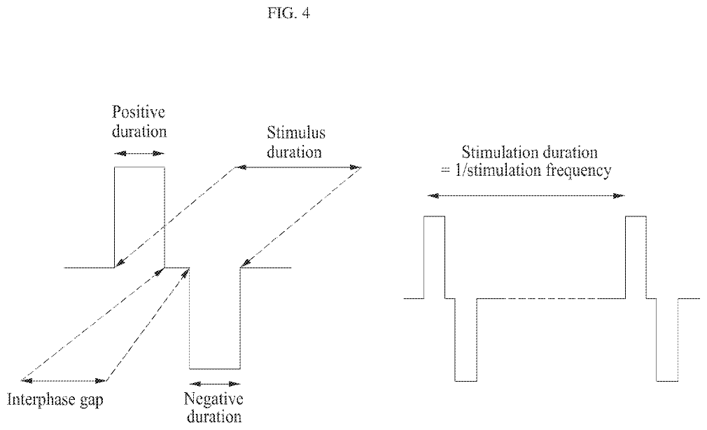

FIG. 4 illustrates an example of stimulus variables that are related to time.

The stimulator controller 230 may adjust (the stimulus) Φ in response to a stimulus generation signal. For example, the time when (the level of the stimulus) Φ₁ is high and (the level of the stimulus) Φ₂ is low may become positive duration. The time when both (the level of the stimulus) Φ₁ and (the level of the stimulus) Φ₂ are low may become an inter-phase gap.

The stimulus frequency dithering range setting unit 210 may change a stimulus frequency by changing variables related to stimulus duration that has been designated by the stimulus characteristic setting unit 220. For example, the stimulus frequency dithering range setting unit 210 may adjust a stimulus frequency by changing the stimulus frequency in a way to add or subtract a stimulus duration variable by a random number, after generating the random number by using a linear feedback shift register (LFST).

Unlike in the above example, if the stimulator controller 230 is constructed in a form such as a digital to analog converter (DAC), an arbitrary waveform, such as a triangle wave or a sine wave, in addition to a bi-phasic square wave may be implemented by changing the amplitude of a signal through a change of a digital value.

In other words, according to the present embodiments, a signal that controls a circuit which may be used to generate a stimulus, such as a current DAC or an LC resonance circuit, including the H-bridge circuit, may be changed so that a stimulus frequency can be changed in a predetermined range on the basis of a center frequency thereof.

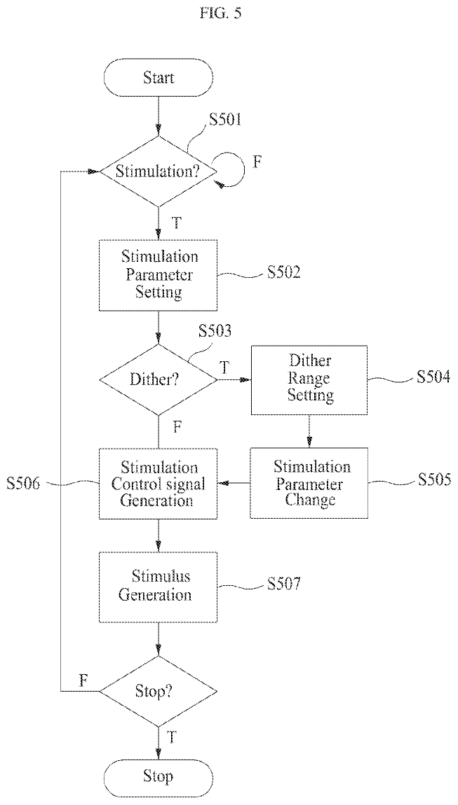

FIG. 5 is a flowchart illustrating an example of the method of removing stimulus artifacts, which may be performed by the computer device according to an embodiment of the present disclosure.

Referring to FIG. 5, the processor 120 may set stimulation parameters in response to a request to generate a stimulus (S501 to S502).

When dithering operates for the setting of a stimulus frequency, the processor 120 may set a stimulus frequency dithering range on the basis of the center frequency of the stimulus frequency and then change stimulation parameters based on the stimulus frequency dithering range (S503 to S505).

The processor 120 may generate a stimulus control signal corresponding to the stimulation parameters, and may generate an actual stimulus in response to the stimulus control signal (S506 and S507).

The processor 120 may generate a stimulus generation signal in which stimulus frequency dithering is changed within a set range in the center frequency, and may then generate an actual stimulus based on the stimulus generation signal and a stimulus waveform setting value.

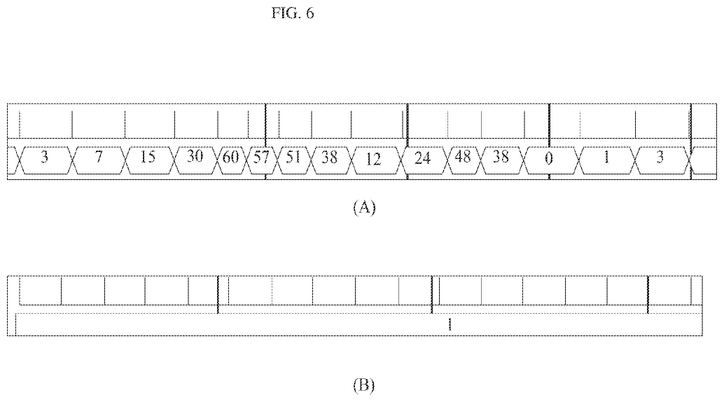

FIGS. 6(A) and 6B) illustrate examples of the results of Verilog simulation experiments. A graph on the upper side in each of FIGS. 6(A) and 6(B) indicates stimulus timing. A graph on the lower side in each of FIGS. 6(A) and 6(B) indicates stimulus timing-random change variables. As the number of the stimulus timing random-change variable is increased, a stimulus frequency is increased. When a dithering function is turned off, stimuli having the same gap appear as in FIG. 6(B). It is expected that when such a stimulus is given, a stimulus target will show a reaction similar to that when a fixed frequency stimulus is given. It is expected that a reduction of a stimulus effect attributable to adaptation is also reduced.

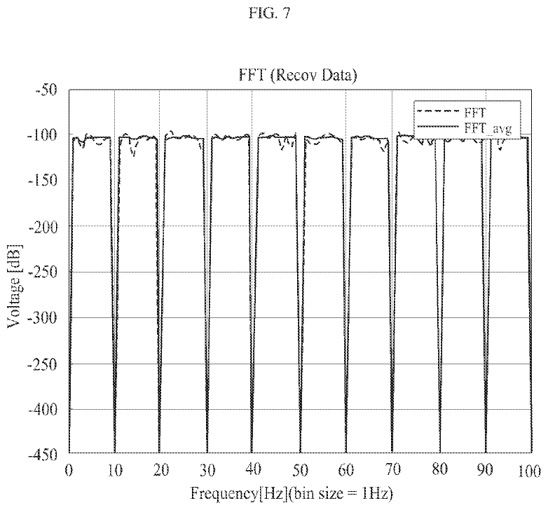

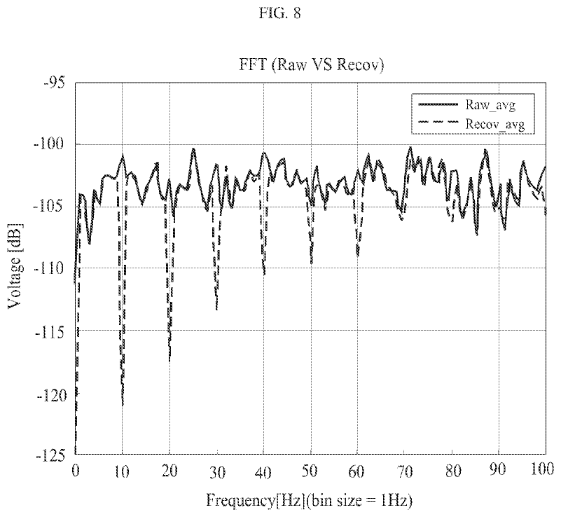

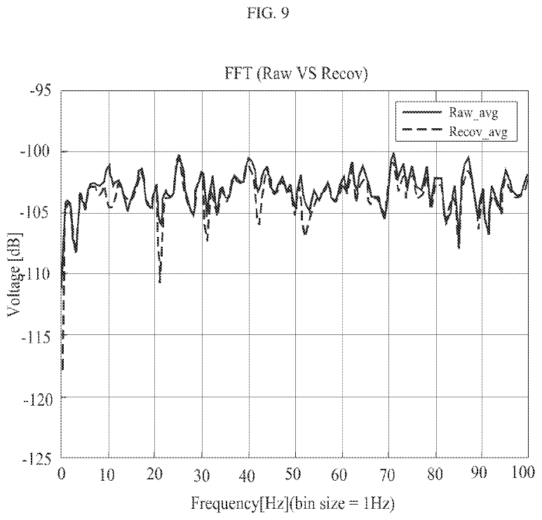

FIGS. 7 to 9 illustrate examples of the results of MATLAB simulations. It may be seen that when frequency dithering is present, a specific frequency attenuation phenomenon that appears when the method of removing stimulus artifacts using a time domain average value is used can be avoided. Conditions for the MATLAB simulations include <64 k sampling, a 10 Hz gap average (6400 samples per interval)> and that dithering is 0% (FIG. 7), 1% (FIG. 8), and 10% (FIG. 9) from the left side. A signal that is used for the MATLAB simulations is a random noise signal that can be easily compared because the entire frequency interval has the same power on average. The graphs illustrate that data after values of several random noise signal are averaged in the time domain was each subjected to a fast Fourier transform (FFT) and then averaged in the frequency domain. It can be seen that if dithering proceeds, the frequency attenuation phenomenon disappears.

As described above, according to embodiments of the present disclosure, it is possible to effectively solve a problem in that a bio signal, such as a stimulus frequency, is also removed when artifacts are removed while maintaining effects of a stimulus by controlling the stimulus frequency to be changed within an intended dithering range. The limit of a technology for removing stimulus artifacts using a time domain average method can be overcome. The technology for removing stimulus artifacts using a time domain average method can be applied to a wider region. A phenomenon in which stimulus efficiency is reduced due to neurolysis attributable to additionally repeated stimuli can also be handled.

The aforementioned device may be implemented as a hardware component, a software component, and/or a combination of a hardware component and a software component. For example, the device and component described in the embodiments may be implemented by using one or more general-purpose computers or special-purpose computers, such as a processor, a controller, an arithmetic logic unit (ALU), a digital signal processor, a microcomputer, a field programmable gate array (FPGA), a programmable logic unit (PLU), a microprocessor, or any other device capable of executing or responding to an instruction. The processing device may perform an operating system (OS) and one or more software applications that are executed on the OS. Furthermore, the processing device may access, store, manipulate, process, and generate data in response to the execution of software. For convenience of understanding, one processing device has been illustrated as being used, but a person having ordinary knowledge in the art may understand that the processing device may include a plurality of processing elements and/or a plurality of types of processing elements. For example, the processing device may include a plurality of processors or one processor and one controller. Furthermore, another processing configuration, such as a parallel processor, is also possible.

Software may include a computer program, a code, an instruction or a combination of one or more of them, and may configure a processing device so that the processing device operates as desired or may instruct the processing devices independently or collectively. The software and/or the data may be embodied in any type of machine, component, physical device, or computer storage medium or device in order to be interpreted by the processing device or to provide an instruction or data to the processing device. The software may be distributed to computer systems that are connected over a network, and may be stored or executed in a distributed manner. The software and the data may be stored in one or more computer-readable recording media.

The method according to an embodiment may be implemented in the form of a program instruction executable by various computer means, and may be stored in a computer-readable medium. In this case, the medium may continue to store a program executable by a computer or may temporarily store the program for execution or download. Furthermore, the medium may be various recording means or storage means having a form in which one or a plurality of pieces of hardware has been combined. The medium is not limited to a medium that is directly connected to a computer system, but may be ones that are distributed and present in a network. Examples of the medium may be magnetic media such as a hard disk, a floppy disk, and a magnetic tape, optical media such as CD-ROM and a DVD, magneto-optical media such as a floptical disk, and ones configured to store a program command, including ROM, RAM, and a flash memory. Furthermore, examples of another medium may include an app store in which apps are distributed, a site in which other various pieces of software are supplied or distributed, and recording media and/or storage media that are managed in a server.

As described above, although the embodiments have been described in connection with the limited embodiments and the drawings, those skilled in the art may modify and change the embodiments in various ways from the description. For example, proper results may be achieved although the aforementioned descriptions are performed in order different from that of the described method and/or the aforementioned elements, such as the system, configuration, device, and circuit, are coupled or combined in a form different from that of the described method or replaced or substituted with other elements or equivalents.

Accordingly, other implementations, other embodiments, and the equivalents of the claims fall within the scope of the claims.

1. A method of removing stimulus artifacts by a computer device comprising at least one processor, the method comprising:
setting, by the at least one processor, a dithering range of a stimulus frequency for generating a stimulus; and
controlling, by the at least one processor, the stimulus frequency to be changed within the dithering range.

2. The method of claim 1, wherein the controlling of the stimulus frequency comprises changing the stimulus frequency by changing variables related to stimulation duration.

3. The method of claim 1, wherein the controlling of the stimulus frequency comprises controlling the stimulus frequency to be changed in a predetermined range on the basis of a center frequency by changing an adjustment signal for generating a signal that turns on/off switches of a stimulus generation circuit as a stimulus signal.

4. The method of claim 1, further comprises setting, by the at least one processor, a center frequency that is a dithering criterion.

5. The method of claim 4, wherein the setting of the dithering range comprises setting the dithering range of the stimulus frequency as a predetermined range on the basis of the center frequency.

6. The method of claim 4, wherein when the stimulus frequency is dithered, the center frequency becomes the dithering criterion so that a frequency dithered within a predetermined range is placed in the stimulus frequency.

7. The method of claim 4, wherein the setting of the center frequency comprises setting at least one of stimulation parameters comprising a form and length of the stimulus.

8. The method of claim 7, wherein the length of the stimulus is set for each phase and comprises an inter-phase gap.

9. A non-volatile computer-readable recording medium in which a computer program for executing the method of removing stimulus artifacts according to claim 1 in the computer device has been stored.

10. A computer device comprising:
at least one processor configured to execute a computer-readable instruction,
wherein the at least one processor comprises:
a dithering range setting unit configured to set a dithering range of a stimulus frequency for generating a stimulus; and
a stimulus characteristic setting unit configured to set variables related to the stimulus so that the stimulus frequency is changed within the dithering range.

11. The computer device of claim 10, wherein the stimulus characteristic setting unit
sets a center frequency that is a dithering criterion, and
sets at least one of stimulation parameters comprising a form and length of the stimulus.

12. The computer device of claim 11, wherein:
the dithering range setting unit sets the dithering range of the stimulus frequency as a predetermined range on the basis of the center frequency, and
when the stimulus frequency is dithered, the center frequency becomes the dithering criterion so that a frequency dithered within the predetermined range is placed in the stimulus frequency.

13. The computer device of claim 11, wherein the length of the stimulus is set for each phase and comprises an inter-phase gap.

14. The computer device of claim 10, wherein the dithering range setting unit changes the stimulus frequency by changing variables related to stimulus duration that has been set by the stimulus characteristic setting unit.

15. The computer device of claim 10, wherein:
the at least one processor further comprises a stimulator controller comprising a stimulus generation circuit, and
the dithering range setting unit changes an adjustment signal for generating a signal that turns on/off switches of the stimulus generation circuit so that the stimulus frequency is changed in a predetermined range on the basis of a center frequency.
